(19) United States
(12) Patent Application Publication
(10) Pub. No.: US 2026/0256395 A1
(43) Pub. Date: Sep. 3, 2026

(54) SYSTEMS AND METHOD FOR LINKING COSMETIC LIP COLORS TO EMOTIONS

(71) Applicants: L'Oreal, Paris (FR); NEWTONE COLORIMETRIE, Lyon (FR)

(72) Inventors: Veronika MAREK, NY, NY (US); Charlotte ROLLAND, Collonges au mont d’or (FR); Olfa BCHIR, Paris (FR); Aurélie COUBART, Elancourt (FR); Anke HADASCH, Paris (FR)

(73) Assignee: L'Oreal, Paris (FR)

(21) Appl. No.: 19/549,968

(22) Filed: Feb. 25, 2026

Related U.S. Application Data
(60) Provisional application No. 63/765,368, filed on Feb. 28, 2025.

(30) Foreign Application Priority Data
Apr. 14, 2025 (FR) FR2503966

(51) Int. Cl. A61B 5/16; A61B 5/00; A61B 5/0533; A61B 5/28; A61B 5/291; A61B 5/318; A61B 5/378; A61B 5/38

(52) U.S. Cl. CPC A61B 5/165; A61B 5/0533; A61B 5/163; A61B 5/28; A61B 5/291; A61B 5/318; A61B 5/378; A61B 5/38; A61B 5/748; A61B 5/7435; A61B 5/744

(57) ABSTRACT

A computer system receives sensor data from sensors configured to measure a user's neurophysiological response to verbal stimuli in the form of an identified emotion presented to the user and visual stimuli in the form of cosmetic colors presented to the user via a user interface; displays representations of available cosmetic colors to the user; receives user input indicating selection of a candidate cosmetic color from among the available cosmetic colors displayed; displays to the user an avatar wearing a virtual cosmetic of the candidate cosmetic color; receives user rating data responsive to display of the avatar wearing the virtual cosmetic of the candidate cosmetic color; and determines whether the candidate cosmetic color is associated with the identified emotion based on the user rating data and the sensor data. The sensors may include brain activity, electrodermal activity, eye tracking, and/or heart activity sensors.

### CROSS-REFERENCES TO RELATED APPLICATIONS

This application claims the benefit of and priority to U.S. Provisional Application No. 63/765,368, filed Feb. 28, 2025, and French Patent Application No. 2503966, filed Apr. 14, 2025, the disclosures of which are incorporated herein by reference.

### SUMMARY

In one aspect, a system comprises a sensor system including sensors configured to obtain sensor data indicative of a user's neurophysiological response to verbal stimuli in the form of an identified emotion presented to the user and visual stimuli in the form of cosmetic colors (e.g., lipstick colors) and an avatar presented to the user via a user interface; and a computer system including computational circuitry configured to cause representations of available cosmetic colors to be displayed to the user via the user interface; computational circuitry configured to receive user input indicating selection of a candidate cosmetic color from among the available cosmetic colors; computational circuitry configured to cause the avatar to be displayed to the user, the avatar wearing a virtual cosmetic of the candidate cosmetic color; computational circuitry configured to receive user rating data responsive to display of the avatar wearing the virtual cosmetic of the candidate cosmetic color; and computational circuitry configured to determine whether the candidate cosmetic color is associated (e.g., positively or negatively, strongly or weakly) with the identified emotion based on the user rating data and the sensor data.

In some embodiments, the user interface displays to the user representations of the available cosmetic colors in cosmetic color families, and the computer system further includes computational circuitry configured to receive user input indicating selection of a cosmetic color family or category. In some embodiments, upon selection of the cosmetic color family or category, the displayed set of available cosmetic colors are updated such that they belong to the selected cosmetic color family or category. The available color families or categories for selection may include traditional color groups that include related shades, or more fanciful or curated color categories, such as colors associated with a particular designer or brand, or colors that are not traditionally associated with a particular cosmetic (e.g., blue or green for lip color). In some embodiments, for lip color, the cosmetic color families include red, pink, purple, orange, and brown.

In some embodiments, the virtual cosmetic is lipstick worn on a lip area of the avatar.

In some embodiments, the user interface displays to the user representations of a plurality of available avatars, and the computer system further includes computational circuitry configured to receive user input indicating selection of the avatar from among the available avatars.

In some embodiments, the user interface displays to the user the identified emotion during display of the avatar wearing the virtual cosmetic of the candidate cosmetic color.

In some embodiments, the available cosmetic colors are determined by clustering colors in a database with a K-means algorithm into clusters of color patches for each of a plurality of color families.

In some embodiments, the sensors include one or more brain activity sensors (e.g., electroencephalogram (EEG) 20-sensors), electrodermal activity (EDA) sensors (e.g., galvanic skin response (GSR) sensors); an eye tracking device configured to detect gaze direction of the user; heart activity sensors (e.g., electrocardiogram (ECG) sensors), or a combination of multiple types of sensors.

In another aspect, a method performed by a computer system comprises receiving sensor data from sensors configured to measure a user's neruophysiological response to verbal stimuli in the form of an identified emotion presented to the user and visual stimuli in the form of cosmetic colors presented to the user via a user interface; displaying representations of available cosmetic colors to the user via the user interface; receiving user input indicating selection of a candidate cosmetic color from among the available cosmetic colors displayed in the user interface; displaying to the user an avatar wearing a virtual cosmetic of the candidate cosmetic color; receiving user rating data responsive to display of the avatar wearing the virtual cosmetic of the candidate cosmetic color; and determining whether the candidate cosmetic color is associated with the identified emotion based on the user rating data and the sensor data.

In some embodiments, the method further comprises displaying cosmetic color families or categories including the representations of the available cosmetic colors; and receiving user input indicating user selection of a cosmetic color family or category from among the displayed cosmetic color families or categories, wherein the displayed representations of the available cosmetic colors belong to the selected cosmetic color family or category. In some embodiments, the method further comprises, responsive to user selection of the cosmetic color family or category, updating the displayed set of available cosmetic colors such that each of the available cosmetic colors in the displayed set belongs to the selected cosmetic color family or category. In some embodiments, the method further comprises displaying to the user representations of a plurality of available avatars; and receiving user input indicating user selection of the avatar from among the available avatars.

This summary is provided to introduce a selection of concepts in a simplified form that are further described below in the Detailed Description. This summary is not intended to identify key features of the claimed subject matter, nor is it intended to be used as an aid in determining the scope of the claimed subject matter.

### BRIEF DESCRIPTION OF THE DRAWINGS

The foregoing aspects and many of the attendant advantages of the disclosed subject matter will become more readily appreciated as the same become better understood by reference to the following detailed description, when taken in conjunction with the accompanying drawings, wherein:

FIG. 1 is a block diagram of a system in which various aspects of the present disclosure may be implemented;

FIGS. 2-4 are screenshot diagrams of aspects of a user interface, according to various aspects of the present disclosure;

FIG. 5 is a flowchart that illustrates an example embodiment of a method of determining whether a cosmetic color is associated with an identified emotion; and

FIG. 6 is a block diagram that illustrates aspects of an exemplary computing device appropriate for use as a computing device of the present disclosure.

### DETAILED DESCRIPTION

Today, consumers express a clear desire for cosmetics that extend beyond mere functionality, seeking products that also uplift their mood and assist to manage their emotions. Although cognitive and color sciences have been used to study connections between emotions and colors, research studies linking emotions to colors have mainly been conducted via questionnaires and surveys on preferences, and no technology has been developed to determine links between emotions and color cosmetics. New and novel technologies to determine relationships between make-up colors and emotions is therefore desirable to create emotionally engaging cosmetic portfolios.

Embodiments described herein provide technical solutions to one or more of the technical problems described above, or other technical problems. In the present disclosure, systems, devices, methods, and processes to link make-up colors, presented as separate color patches and as simulations on digital models, to positive emotions are described.

A cognitive and color science research study was performed to decode the link between positive make-up related emotions and digital make-up lip colors. The study consisted of presenting digital color stimuli to consumers of diverse ethnicities, ages, and skin tones, and evaluating their emotional reaction via physiological measurements such as brain activity (e.g., via electroencephalography (EEG)), heart rhythm (e.g., via electrocardiography (ECG)), skin conductance (e.g., via galvanic skin response (GSR)) and visual attention (e.g., via eye tracking), along with verbal responses to questions or prompts. The study participants were 123 American consumers of diverse ethnic backgrounds (White, Afro-American, Latino and Asian), The study sought to identify and measure associations between 15 positive make-up relevant emotions (Relaxed,” “Peaceful,” “Secure,” “Casual,” “Happy,” “Energetic,” “Amazed,” “Playful,” “Intrigued,” “Self-confident,” “Perfect,” “Sophisticated,” “Daring,” “Trendy,” “Sensual”) with colors selected from 168 available colors in 6 color families (Red, Pink, Coral, Purple, Brown, and Other).

The available colors were selected from a database of 7965 shades in an L*a*b* color space. This database included 3540 solid lipstick vitro application colors, 925 liquid lipstick vitro application colors, and 3500 colors from physical swatches. The colors in the database were classified by color family (Red, Pink, Coral, Purple, Brown, and Other) and sub-family (e.g., undertone/hue (Cool, Neutral, Warm), lightness (Light, Medium, Dark), and chroma (High, Medium, Low)). The colors in the database were then clustered with a K-means algorithm into clusters of 25 color patches for each of six color families. K-means is an unsupervised machine learning algorithm, an iterative algorithm that tries to partition the dataset into K pre-defined distinct non-overlapping subgroups (clusters) where each data point belongs to only one group. Market information and expert consultation was then used to add new and high-performing shades (by market information) in each color family to complete the set of available colors.

The available colors were presented in a user interface as digital square patches and simulation on lips of an avatar (virtual try-on—VTO), displayed on a color calibrated screen. User input in the form of verbal answers were obtained, along with sensor data to measure visual attention (eye tracking), skin conductance (GSR), heart rhythm (ECG) and brain activity (EEG).

As the selection and evaluation process continued, real-time sensor data was gathered from the participant via sensors, namely eye tracking, GSR, ECG, and EEG sensor data.

In this illustrative process, a baseline of sensor data was obtained by evaluating participants'responses to displayed images that are known to provoke different emotional reactions, such as pictures of a smiling baby, a snake, a kitten, a sunset, and a crying person. Next, a positive emotion was chosen from among a list of 15 such emotions (e.g., “playful,” “relaxed,” “secure,” “self-confident,” “daring”), and the participant was asked to list three words that the participant associated with that emotion (e.g., “joy,” “calm,” and “peaceful” for “relaxed”; “bold,” “relaxed,” and “adventurous” for “daring”). This step was designed to put the user in the identified mood/emotion as the user considered the particular emotion being evaluated.

Color boards were generated in a semi-randomized fashion (24 boards (four per color family) for each of 15 emotions), with each color board including a set of color patches. For each participant, a first set of square color patches was selected from among the available boards for that emotion and presented to the participant, who was asked to select one or two color families they associated with that emotion. In this first set, a smaller number of patches is presented for each color family (e.g., five for each color family). Then, a second set of color patches (e.g., 25 or 30) was presented to the participant based on each of the selected color families for that emotion. The boards were semi-randomized by sorting the colors for each family on each board in different ways (e.g., lighter-to-darker by row, lighter-to-darker by column, darker-to-lighter by row, darker-to-lighter by column). The presentation of patches for selection used a grid pattern with a background color selected to have minimal impact on the appearance of the colors in those patches, for example, by ensuring that the background was a neutral/gray color that was lighter than the lightest color patch. In addition, the square color patches when presented were separated by a sufficient distance to reduce the potentially distracting effect of a Hermann grid illusion, in which a grid of colored squares on a light background causes human eyes to perceive faint figures at the intersections of the light-colored background lines.

The participant was asked to select one to three of those patches from the second set as lipstick shades associated with the emotion being evaluated. The participant was assigned an avatar from a set of five photorealistic avatars to use as a virtual model. The selected avatar was presented to the participant in a virtual try-on (VTO) interface with lipstick shades for each of the one to three selected color patches having been virtually applied to the avatar. The participant was then asked to rate the desirability of each presented lipstick shade. In one approach, the desirability was rated verbally on a scale of 0 to 5, with 5 indicating the highest level of desirability, in different categories of desirability (e.g., liking the lipstick shade, likelihood/intention of wearing the lipstick shade, likelihood/intention of buying the lipstick shade).

For each emotion, acquired data facilitated building color sets for cosmetics that were chosen verbally and also provoked a statistically significant positive physiological reaction. As noted above, the physiological reaction was recorded as sensor data during the whole above-described process of color choice and evaluation. Emotional impact was determined based on three dimensions (valence (positive, neutral, negative), intensity, and duration) using measures obtained from the sensor data. With this data, results were obtained for different ethnicities, ages, and skin tones.

This study observed how emotional context might impact lip make-up color preferences. The results suggest new possibilities to create emotionally engaging lip shade portfolios and thus a better consumer experience. By using sensor data in combination with users'expressed preferences via user input data, colors can be associated with particular emotions at a high level of confidence, and situations where a participant's expressed affinity for a particular color (as expressed in user input data) as being associated with a particular emotion is not aligned with the implicit emotional responses indicated by the sensor data can be accounted for. For example, an understanding of which colors were “liked” but did not provoke any significant physiological emotional reaction was obtained.

Specifically, this study indicated that only 55% of verbal choices of color patches are confirmed as desirable (e.g., rating of 4 or 5 out of 5) when applied to lips of an avatar in a VTO interface. And of those that are confirmed, only 36% (or about 20% of all color patch choices) are determined to provoke a positive emotion as measured via sensor data. With this information, which may be generated using described embodiments, color sets for cosmetics can be selected that are not only expressed as a preference but also are associated with a strong emotional impact.

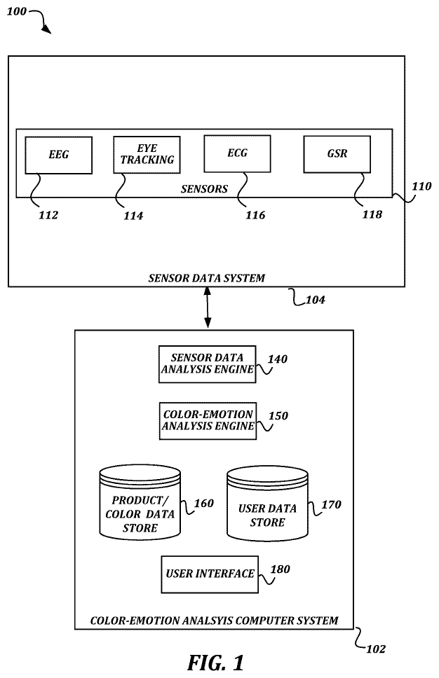

FIG. 1 is a block diagram that illustrates a system 100 in which various aspects of the present disclosure may be implemented. In the illustrative arrangement depicted in FIG. 1, color-emotion analysis computer system 102 receives sensor data from sensor data system 104.

In the illustrated system, physiological responses of a user are measured using sensor data system 104. Sensor data system 104 includes one or more sensors 110. In the example shown in FIG. 1, sensors 110 include EEG sensor module 112, eye tracking module 114, ECG module 116, and GSR module 118. Each sensor module is configured to measure different movements or physiological responses of the user. For example, in EEG sensor module 112, electrical activity of the brain of the user in the form of EEG signals is measured using EEG sensors, in response to a user's exposure to a visual stimulus, such as a displayed color patch or avatar. In an illustrative scenario, the EEG sensors are placed in proximity to various regions of the user's brain, for example via a suitable headset, in order to measure the electrical activity of the corresponding regions of the user's brain.

In some embodiments, sensor data system 104 transmits sensor signals as data to computer system 102 via a network, which may include any suitable wireless communication technology (including but not limited to Wi-Fi, WiMAX, Bluetooth, 2G, 3G, 4G, 5G, and LTE), wired communication technology (including but not limited to Ethernet, USB, and FireWire), or combinations thereof.

Sensor data system 104 includes processing and transmission circuitry, which may be distributed among individual sensors 110 or shared by one or more sensors. For example, in an illustrative configuration, EEG module 112 comprises an EEG headset that includes EEG sensors or electrodes that are configured to measure voltage changes in the user's brain as EEG signals. EEG signals measured by the EEG sensors can be processed by circuitry that is specific to EEG module 112 or shared with other sensor modules in sensor data system 104 for transmission to the computer system 102 for storage, data processing and/or analysis, etc. In an illustrative scenario, EEG signals are amplified by an amplifier and digitized by an A/D converter prior to arrival at a transmitter. In some embodiments, the EEG signals can be filtered in the analog domain prior to conversion by the A/D converter or in the digital domain after conversion by the A/D converter via one or more filters. In some embodiments, filtered signals are sent to a multiplexer before transmission via the transmitter to computer system 102. Similar circuitry can be used to process and transmit signals measured by other sensor modules, such as ECG module 116 or GSR module 118.

Computer system 102 includes one or more computers that implement the illustrated features, e.g., in a cloud computing or other network arrangement. As illustrated in FIG. 1, the computer system 102 includes sensor data analysis engine 140, color-emotion analysis engine 150, product/color data store 160, user data store 170, and user interface 180. Computer system 120 generates color boards based on color data obtained from product/color data store 160 and presents these color boards to a user via user interface 180. Computer system 102 obtains user response data via user interface 180 and sensor data via sensor data system 104.

In some embodiments, sensor data analysis engine 140 is configured to collect sensor data from sensor data system 104, process the sensor data, and record the sensor data in a time-based manner in user data store 170. In some embodiments, processing the sensor data may include but is not limited to converting, filtering, transforming, and/or the like, prior to the further analysis that is performed in color-emotion analysis engine 150.

In some embodiments, computer system 102 includes a display device for presenting features of user interface 180. In other embodiments, a device separate from computer system 102 includes a display device that is used to display features of user interface 180. In any embodiments described herein involving a display device, the display device is any suitable type of display device, including but not limited to an LED display, an OLED display, or an LCD display, that is capable of presenting visual user interfaces to the user. Illustrative examples of such user interfaces are described in more detail below.

User response data obtained from user interface 180 can be stored in user data store 170 for future processing or analysis. Computer system 102 generates color-emotion data with color-emotion analysis engine 150. In some embodiments, color-emotion data includes identified associations between particular colors and particular emotions, with the color-emotion data being generated based on user response data and sensor data. Color-emotion data can then be further processed or transmitted to other computers or systems. In some embodiments, the color-emotion data is used to generate for example, cosmetics routine recommendations or product recommendations, such as recommendations to help users achieve a look that associated with a particular emotion.

Sensor data analysis engine 140 and/or color-emotion analysis engine 150 may employ machine learning or artificial intelligence techniques (e.g., template matching, feature extraction and matching, classification, artificial neural networks, deep learning architectures, genetic algorithms, or the like).

Described embodiments allow for different machine learning approaches, or combinations of approaches, to be employed, using one or more machine learning models. In an illustrative scenario, color-emotion analysis engine 150 includes a machine learning model for determining whether sensor data associated with a particular color indicates a positive emotional response. In some embodiments, the machine learning models are neural networks, including but not limited to feedforward neural networks, convolutional neural networks (CNNs), recurrent neural networks, and generative adversarial networks (GANs).

In an embodiment, neural networks include neurons (nodes), weights, biases, activation functions, layers, and the like. In an embodiment, neurons (nodes) are fundamental processing units where each neuron receives inputs, processes them, and produces an output. In an embodiment, weights include numerical values that represent the strength of the connection between neurons. In an embodiment, the weights are adjusted during training. In an embodiment, biases include additional parameters added to the weighted sum of inputs, allowing the activation function to be shifted. In an embodiment, activation functions include non-linear functions applied to the weighted sum of inputs plus bias. This introduces non-linearity, enabling the network to learn complex patterns. Illustrative activation functions include ReLU (rectified linear units), Sigmoid, Tanh, and the like. In an embodiment, neural networks include neurons organized into layers. Non-limiting examples of layers include input layers configured to receive the raw data; hidden layers between the input and output layers where the bulk of the computation happens; output layers configured to produce the final prediction, result, classification, etc. In some embodiments, any suitable training technique may be used, including but not limited to gradient descent, which may include stochastic, batch, and mini-batch gradient descent.

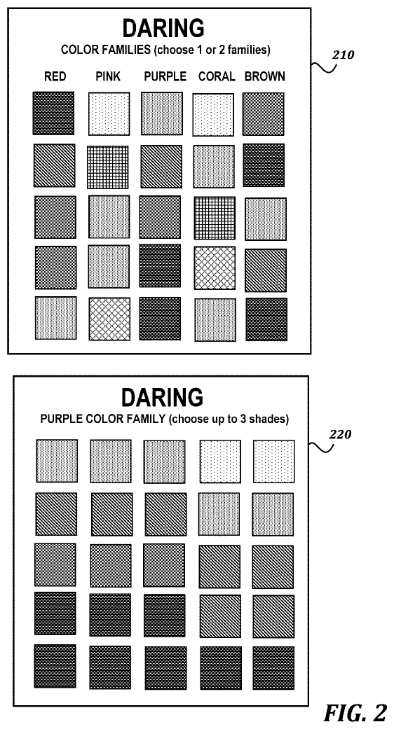

FIGS. 2-4 are screenshot diagrams of aspects of a user interface, according to various aspects of the present disclosure. In some embodiments, these screenshot diagrams are representative of user interface features presented to a user as sensor data is gathered from the user, and after an emotion (“Daring”) has been selected. The illustrative user interface features are presented on a color-calibrated display device in order to display correct colors for selection. FIG. 2 includes a color family selection screen 210, in which a user is prompted to select a color family associated with “Daring.” FIG. 2 also includes a shade selection screen 220 in which the user is prompted to select up to three shades (in the Purple color family, in this example) that the user finds to be representative of “Daring.” In practice, user input indicating these selections may be collected in different ways, such by recognizing taps or other gestures in a corresponding location on a touch screen (e.g., in the Red column to select the Red color family, or on a specific color patch to select that color) or by detecting a verbal response via a microphone, in which the audio signal can be processed with speech recognition software to determine the selection. Although the color patches are not individually labeled in FIG. 2 for ease of illustration, it should be understood that the color patches are identified in some embodiments with unique identifiers (e.g., Orange 2, Red 11, Purple 20) that allow unambiguous identification and selection of the corresponding colors. In some embodiments, these identifiers are displayed in the user interface next to the corresponding color patch, and the identifiers can be recited aloud by the user to allow for verbal selection of the colors.

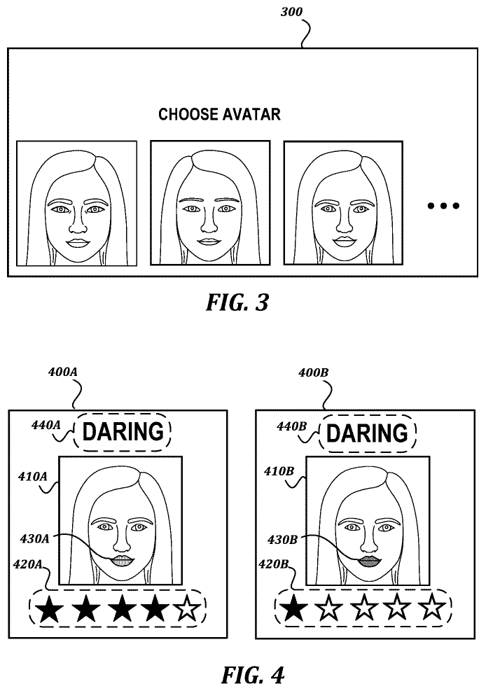

FIG. 3 includes an avatar selection screen 300 in which the user is prompted to select an avatar to use as a virtual model for wearing a cosmetic (e.g., lipstick, eyeshadow, etc.) having the selected color. Although three available avatars are shown for ease of illustration, it should be understood that any number of avatars may be presented for selection. In some embodiments, the avatars are pre-generated and are not based on the appearance of the user.

Alternatively, a custom avatar based in the user's appearance may be used (e.g., based on user images captured at a prior time or concurrently during the data collection process). In such an alternative implementation, the system may include a face detection module that provides facial feature information and calculates one or more regions for modification in a VTO scenario based on user image data provided to the face detection module and an indication of the area in the user's custom avatar on which the cosmetic is to be applied. In an illustrative implementation, a face detection module comprises the face detection application programming interface (API) for ML Kit, available from Google LLC.

Referring now to FIG. 4, the selected avatar is presented to the participant in a virtual try-on (VTO) interface wearing a virtual cosmetic having the selected color(s). In some embodiments, the process is specific to a particular type of cosmetic, such as lipstick.

In screen 400A, the avatar image 410A is shown wearing virtual lipstick 430A having a first selected shade in the lip area, and in screen 400B, the avatar image 410B is shown wearing virtual lipstick 430B having a selected shade in the lip area. The optional rating areas (420A, 420B) represent ratings given by the user for desirability of each presented lipstick shade in terms of its association with a particular emotion, and that emotion (e.g., “Daring”) may optionally be displayed (e.g., in display area 440A, 440B) during the evaluation process. Ratings can be collected in different ways (e.g., verbally or via a touch screen interface). In one approach, the ratings are displayed to the user to allow the user to confirm the rating before proceeding further, regardless of how the rating was collected. In the illustrated example, the desirability is rated on a scale of 0 to 5, with 5 indicating the highest level of desirability (e.g., liking, wearing, buying) and 0 indicating total rejection of the shade relative to the emotion being evaluated.

Thus, the examples shown in FIGS. 1-4 are used in some embodiments to select or recommend colors for cosmetics that are both chosen by the user and associated with a significant positive physiological reaction. Emotional impact can be determined (e.g., by color-emotion analysis engine 150) based on different emotional dimensions (e.g., valence, intensity, and duration) using measures obtained from the sensor data. The results also can be segmented based on different categories, such as ethnicities, ages, and skin tones.

Many alternatives to the arrangements and usage scenarios described herein are possible. For example, functionality described as being performed by a server computer system may instead be performed by a client computing device, or vice versa, or such functionality may be performed by different devices or systems. As another example, functionality described as being performed by a particular module or component may instead be performed by a combination of such modules or components, or by a different module or component, or functionality described as being performed by individual modules or components may be combined in a single module or component.

In general, the word “engine,” as used herein, refers to logic embodied in hardware or software instructions written in a programming language, such as C, C++, COBOL, JAVA™, PHP, Perl, HTML, CSS, JavaScript, VBScript, ASPX, Microsoft .NET™, and/or the like. An engine may be compiled into executable programs or written in interpreted programming languages. Software engines may be callable from other engines or from themselves. Generally, the engines described herein refer to logical modules that can be merged with other engines or divided into sub-engines. The engines can be stored in any type of computer-readable medium or computer storage device and be stored on and executed by one or more general purpose computers, thus creating a special purpose computer configured to provide the engine or the functionality thereof.

As understood by one of ordinary skill in the art, a “data store” as described herein may be any suitable device configured to store data for access by a computing device. One example of a data store is a highly reliable, high-speed relational database management system (DBMS) executing on one or more computing devices and accessible over a high-speed network. Another example of a data store is a key-value store. However, any other suitable storage technique and/or device capable of quickly and reliably providing the stored data in response to queries may be used, and the computing device may be accessible locally instead of over a network, or may be provided as a cloud-based service. A data store may also include data stored in an organized manner on a computer-readable storage medium, as described further below. Separate data stores described herein may be combined into a single data store, and/or a single data store described herein may be separated into multiple data stores, without departing from the scope of the present disclosure.

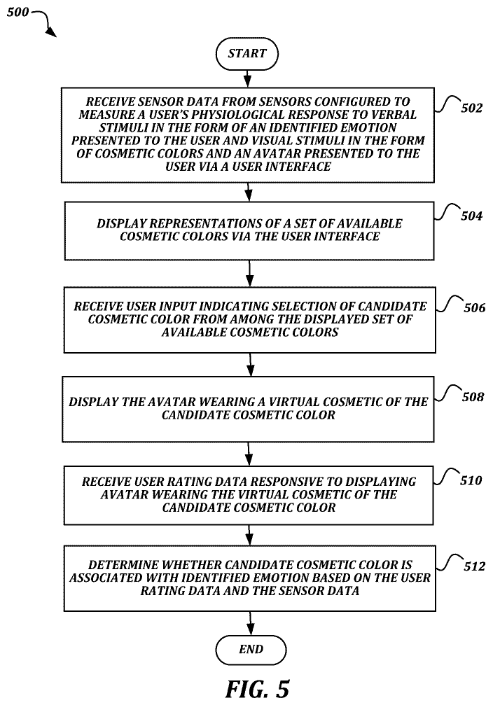

FIG. 5 is a flowchart that illustrates an example embodiment of a method of determining whether a cosmetic color is associated with an identified emotion. As illustrated, the method 500 is implemented by a computer system. The method 500 may be implemented by a server computer system including features of computer system 102, by a client computing device, or a combination thereof, or by some other computing device or system.

From a start block, the method 500 proceeds to block 502, where the computer system receives sensor data from sensors configured to measure a user's physiological response to verbal stimuli in the form of an identified emotion presented to the user and visual stimuli in the form of cosmetic colors presented to the user via a user interface. The method 500 proceeds to block 504, where the computer system displays representations of available cosmetic colors to the user via the user interface. In an illustrative scenario, the available cosmetic colors are determined by clustering colors in a database with a K-means algorithm into clusters of color patches for each of a plurality of color families or categories. In some embodiments, the sensors include a brain activity sensor (e.g., one or more EEG sensors), an electrodermal activity sensor (e.g., one or more GSR sensors), an eye tracking device (e.g., a camera with the user's eyes in its field of few, where the eye tracking data can be processed to determine gaze direction), a heart activity sensor (e.g., one or more ECG sensors), or a combination thereof in which sensors of different types are present or are combined in one or more individual sensor modules.

The method 500 proceeds to block 506, where the computer system receives user input indicating selection of a candidate cosmetic color from among the available cosmetic colors displayed in the user interface. In some embodiments, the method further comprises displaying to the user representations of the available cosmetic colors in cosmetic color families or categories and receiving user input indicating user selection of a cosmetic color family or category from among the displayed cosmetic color families or categories. In some embodiments, the displayed representations of the available cosmetic colors belong to the selected cosmetic color family or category. In some embodiments, for lip color, the displayed cosmetic color families include red, pink, purple, orange, and brown. The available color families or categories for selection may include traditional color groups that include related shades, or more fanciful or curated color categories, such as colors associated with a particular designer or brand, or colors that are not traditionally associated with a particular cosmetic (e.g., blue or green for lip color).

The method 500 proceeds to block 508, where the computer system displays to the user an avatar wearing a virtual cosmetic of the candidate cosmetic color. In some embodiments, the virtual cosmetic is lipstick worn on a lip area of the avatar. In some embodiments, the method further comprises displaying representations of a plurality of available avatars and receiving user input indicating user selection of the avatar from among the available avatars.

The method 500 proceeds to block 510, where the computer system receives user rating data responsive to the display of the avatar wearing a virtual cosmetic of the candidate cosmetic color.

The method 500 proceeds to block 512, where the computer system determines whether the candidate cosmetic color is associated with the identified emotion based on the user rating data and the sensor data.

The techniques described herein can be used in a variety of scenarios. In one illustrative scenario, one or more described techniques are used by a cosmetics manufacturer to obtain information regarding which cosmetic colors are likely to cause a positive emotional reaction in the wearer, which can inform the composition of available colors in a cosmetics product line. In another illustrative scenario, a user can use the system to select a cosmetic color that causes a positive emotional reaction for the user, and this information can then be used to generate recommendations for products to the user. For example, after highly rating an avatar wearing the cosmetic, the system offers the user an option to use a VTO system to virtually try on the cosmetic.

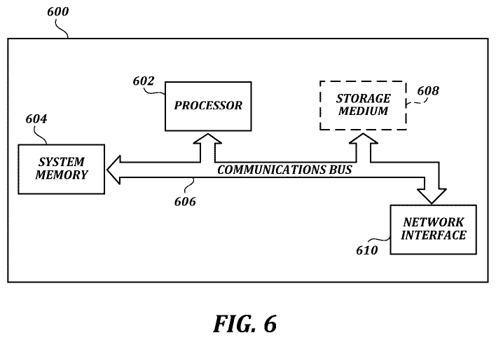

FIG. 6 is a block diagram that illustrates aspects of an exemplary computing device 600 appropriate for use as a computing device of the present disclosure. While multiple different types of computing devices were discussed above, the exemplary computing device 600 describes various elements that are common to many different types of computing devices. While FIG. 6 is described with reference to a computing device that is implemented as a device on a network, the description below is applicable to servers, personal computers, mobile phones, smart phones, tablet computers, embedded computing devices, and other devices that may be used to implement portions of embodiments of the present disclosure. Moreover, the computing device 600 may be any one of any number of currently available or yet to be developed devices.

In its most basic configuration, the computing device 600 includes at least one processor 602 (e.g., one or more central processing units (CPUs) for general purpose tasks and/or graphics processing units (GPUs) for graphics or machine learning or artificial intelligence intensive tasks) and a system memory 604 connected by a communication bus 606. Depending on the exact configuration and type of device, the system memory 604 may be volatile or nonvolatile memory, such as read only memory (“ROM”), random access memory (“RAM”), EEPROM, flash memory, or similar memory technology. System memory 604 typically stores data and/or program modules that are immediately accessible to and/or currently being operated on by the processor 602. In this regard, the processor 602 may serve as a computational center of the computing device 600 by supporting the execution of instructions.

As further illustrated in FIG. 6, the computing device 600 may include a network interface 610 comprising one or more components for communicating with other devices over a network. Embodiments of the present disclosure may access basic services that utilize the network interface 610 to perform communications using common network protocols. The network interface 610 may also include a wireless network interface configured to communicate via one or more wireless communication protocols, such as WiFi, 2G, 3G, 4G, LTE, 5G, WiMAX, Bluetooth, Bluetooth low energy, and/or the like. The network interface 610 illustrated in FIG. 6 may represent one or more wireless interfaces or physical communication interfaces described and illustrated above with respect to particular components of the systems described herein.

In the exemplary embodiment depicted in FIG. 6, the computing device 600 also includes a storage medium 608. However, services may be accessed using a computing device that does not include means for persisting data to a local storage medium. Therefore, the storage medium 608 depicted in FIG. 6 is represented with a dashed line to indicate that the storage medium 608 is optional. In any event, the storage medium 608 may be volatile or nonvolatile, removable or nonremovable, implemented using any technology capable of storing information such as, but not limited to, a hard drive, solid state drive, CD ROM, digital versatile disk (DVD), or other disk storage, magnetic cassettes, magnetic tape, magnetic disk storage, and/or the like.

As used herein, the term “computer-readable medium” includes volatile and non-volatile and removable and non-removable media implemented in any method or technology capable of storing information, such as computer readable instructions, data structures, program modules, or other data. In this regard, the system memory 604 and storage medium 608 depicted in FIG. 6 are examples of computer-readable media.

Suitable implementations of computing devices that include a processor 602, system memory 604, communication bus 606, storage medium 608, and network interface 610 are known and commercially available. For ease of illustration and because it is not important for an understanding of the claimed subject matter, FIG. 6 does not show some of the typical components of many computing devices. In this regard, the computing device 600 may include input devices, such as a keyboard, keypad, mouse, microphone, touch input device, touch screen, tablet, and/or the like. Such input devices may be coupled to the computing device 600 by wired or wireless connections including radio frequency (RF), infrared, serial, parallel, Bluetooth®, Bluetooth® low energy, USB, or other suitable connections protocols using wireless or physical connections. Similarly, the computing device 600 may also include output devices such as a display, speakers, printer, etc.

In an embodiment, during operation, one or more embodiments of the disclosed technologies and methodologies include computational circuitry configured to generate a virtual makeup simulation created based on a user-selected target emotion and one or more parameters associated with a detected physiological response to a curated color set, shade set, tint set, or the like, or combinations thereof.

In an embodiment, the term color is defined as a property possessed by an object producing different sensations on the eye because of the way the object reflects or emits light. In an embodiment, a color comprises a specific combination of hue, saturation, and lightness or brightness. In an embodiment, the tint of a color is a lighter version of that color, and a shade is a darker version. In an embodiment, a tint is produced by adding white to a base hue, resulting in a lighter, paler, or pastel color. In an embodiment, a shade is produced by adding black, creating a darker, deeper version. In an embodiment, the term tone is used to describe the lightness (tint) or darkness (shade) of a basic color. In an embodiment, tone is a more muted version created by adding gray (or both black and white) to a color, reducing its intensity. In an embodiment, color sets, shade sets, tint sets, and the like are used to create cohesive palettes, such as monochromatic, analogous, complementary, and the like.

In an embodiment, a color set includes one or more color family sets. In an embodiment, a color family comprises a set of hues with similar undertones (e.g., cool blues, warm reds, natural browns, and the like). Non-limiting examples of color sets include blue family color set, green family color set, neutral family color set, red family color set, and the like. Further non-limiting examples of color sets include temperature-based families. In an embodiment, temperature-based families are characterized by the emotions or physical sensations they evoke. For example, in an embodiment, warm colors like reds, oranges, yellows, and the like are reminiscent of the sun, fire, feelings of energy, passion, excitement, and the like. In an embodiment, cool colors like blues, greens, purples, and the like are reminiscent of water, nature, a sense of calm, serenity, relaxation, and the like. In an embodiment, neutral colors like whites, blacks, grays, browns, and the like are often used as backgrounds because they do not lean heavily toward warm or cool. In an embodiment, a color set includes composition-based families based on primary colors like red, yellow, or blue, and secondary colors like orange (red+yellow), green (blue+yellow), and purple/violet (red+blue).

In an embodiment, a color set includes one or more thematic families. Non-limiting examples of thematic families include earth tones comprising colors found in nature like umber, ochre, terracotta, and moss green; pastels/tints comprising softened colors created by adding white to a pure hue, such as lavender, mint, pale pink, and the like; and bold/vibrant tones comprising highly saturated, eye-catching hues like electric blue, hot pink, bright yellow; or the like.

In an embodiment, a shade set comprises a range of a single color darkened by adding black, ranging from slightly darker to nearly black. In an embodiment, a shade set includes one or more shades of a color family. Non-limiting examples of shade sets include (navy, cobalt, royal blue, midnight blue), (forest green, olive, emerald, sage), (charcoal, cool gray, warm gray, beige), (maroon, burgundy, crimson, blood red) and the like.

In an embodiment, the disclosed technologies and methodologies described herein include a makeup palette generation system. In an embodiment, the makeup palette generation system includes circuitry configured to generate one or more virtual objects forming part of color set, a shade set, a tint set, or a tone set on a command line user interface, a display, a graphical user interface, a menu driven user interface, a touch-sensitive display, a user input device, a user interface, or a system of interactive visual components.

In an embodiment, the system includes circuitry configured to acquire user-specific neurophysiological data responsive to the generated one or more virtual objects forming part of color set, a shade set, a tint set, or a tone set. In an embodiment, the system includes circuitry configured to generate a makeup palette responsive to at least inputs associated with a user-selected target emotion and the acquired user-specific neurophysiological response data. In an embodiment, the system includes circuitry configured to generate a makeup simulation on an avatar based on the makeup palette generated responsive to the at least inputs associated with the user-selected target emotion and the acquired user-specific neurophysiological response data.

In an embodiment, the circuitry configured to acquire user-specific neurophysiological data includes circuitry configured to acquire one or more of auditory response signals, visual attention (eye tracking) measurements, skin conductance (GSR) transducer data, heart rhythm (ECG) transducer data, brain activity (EEG) transducer data, and the like.

In an embodiment, the circuitry configured to acquire user-specific neurophysiological data includes circuitry configured to acquire user-specific neurophysiological response data responsive to the generated one or more objects forming part of the personality attribute set. In an embodiment, the circuitry configured to acquire user-specific neurophysiological data includes circuitry configured to acquire user-specific neurophysiological response data responsive to presenting cosmetic colors and an avatar to the user via a command line user interface, a display, a graphical user interface, a menu driven user interface, a touch-sensitive display, a user input device, a user interface, a system of interactive visual components, or the like. In an embodiment, during operation, the technologies and methodologies described herein are configured to generate an interactive avatar having one or more physical attributes inferred from a target emotion attribute set.

In an embodiment, during operation, the technologies and methodologies described herein are configured to generate an interactive avatar on one or more client devices, the interactive avatar having one or more physical attributes inferred from a color set, a shade set, a tint set, a tone set, or the like. In an embodiment, during operation, the technologies and methodologies described herein are configured to generate an interactive avatar on one or more client devices, the interactive avatar having one or more physical attributes inferred from a target emotion profile. In an embodiment, during operation, the technologies and methodologies described herein are configured to generate a makeup palette responsive to a user-selected target emotion profile.

In an embodiment, during operation, the technologies and methodologies described herein are configured to generate a makeup palette responsive to a user-selected color set and target emotion. In an embodiment, during operation, the technologies and methodologies described herein are configured to generate a makeup palette responsive to a user-selected shade set and target emotion. In an embodiment, during operation, the technologies and methodologies described herein are configured to generate a makeup palette responsive to a user-selected tint set and target emotion. In an embodiment, during operation, the technologies and methodologies described herein are configured to generate a makeup palette responsive to a user-selected color set, shade set, tint set, or the like.

In an embodiment, during operation, the technologies and methodologies described herein are configured to generate a makeup palette responsive to one or more of a user-selected color set, a user-selected shade set, a user-selected tint set, a user-selected target emotion, or the like, or combinations thereof. In an embodiment, during operation, the technologies and methodologies described herein are configured to generate one or more virtual objects on a display associated with a user-selected color set, a user-selected shade set, a user-selected tint set, a user-selected target emotion, or the like, or combinations thereof. In an embodiment, during operation, the technologies and methodologies described herein are configured to generate a makeup simulation on an avatar based on a makeup palette generated responsive to one or more parameters associated with a temperature-based color family.

In an embodiment, the makeup palette generation system includes electrical circuitry including a hardware processor, a display, and a graphical user interface (GUI) configured to render one or more of a user-selected color set, a user-selected shade set, a user-selected tint set, a user-selected target emotion, or the like, or combinations thereof. In an embodiment, the makeup palette generation system can be implemented such as, for example, by generating a virtual environment capable of enabling interaction between one or more users and a makeup simulation on an avatar.

In an embodiment, the makeup palette generation system includes one or more components having electrical circuitry configured to generate (determine, assess, calculate, predict, derive, or the like) one or more instances of a color set, shade set, tint set, or the like on a virtual display, a user interface, a command line user interface, a menu driven user interface, a graphical user interface, a display, a user input device, a system of interactive visual components, or the like.

In an embodiment, the makeup palette generation system includes one or more components having electrical circuitry configured to generate (determine, assess, calculate, predict, derive, or the like) the neurophysiological state of a user during interaction with the system. For example, in an embodiment, the makeup palette generation system includes sensors configured to obtain sensor data indicative of a user's neurophysiological response to verbal stimuli in the form of an identified emotion presented to the user and visual stimuli in the form of cosmetic colors and an avatar presented to the user via a user interface.

In an embodiment, electrical circuitry includes, among other things, one or more computing devices such as a processor (e.g., a microprocessor, a quantum processor, qubit processor, etc.), a central processing unit (CPU), a digital signal processor (DSP), an application-specific integrated circuit (ASIC), a field programmable gate array (FPGA), and the like, or any combinations thereof, and can include discrete digital or analog circuit elements or electronics, or combinations thereof. In an embodiment, electrical circuitry includes one or more ASICs having a plurality of predefined logic components. In an embodiment, electrical circuitry includes one or more FPGAs, each having a plurality of programmable logic components.

In an embodiment, electrical circuitry includes one or more electric circuits, printed circuits, flexible circuits, electrical conductors, electrodes, cavity resonators, conducting traces, ceramic patterned electrodes, electro-mechanical components, transducers, and the like.

In an embodiment, electrical circuitry includes one or more components operably coupled (e.g., communicatively, electromagnetically, magnetically, ultrasonically, optically, inductively, electrically, capacitively coupled, wirelessly coupled, and the like) to each other. In an embodiment, electrical circuitry includes one or more remotely located components. In an embodiment, remotely located components are operably coupled, for example, via wireless communication. In an embodiment, remotely located components are operably coupled, for example, via one or more communication modules, receivers, transmitters, transceivers, and the like.

In an embodiment, electrical circuitry includes memory that, for example, stores instructions or information. Non-limiting examples of memory include volatile memory (e.g., Random Access Memory (RAM), Dynamic Random Access Memory (DRAM), and the like), non-volatile memory (e.g., Read-Only Memory (ROM), Electrically Erasable Programmable Read-Only Memory (EEPROM), Compact Disc Read-Only Memory (CD-ROM), and the like), persistent memory, and the like. Further non-limiting examples of memory include Erasable Programmable Read-Only Memory (EPROM), flash memory, and the like. In an embodiment, memory is coupled to, for example, one or more computing devices by one or more instructions, information, or power buses.

In an embodiment, electrical circuitry includes one or more computer-readable media drives, interface sockets, Universal Serial Bus (USB) ports, memory card slots, and the like, and one or more input/output components such as, for example, a graphical user interface, a display, a keyboard, a keypad, a trackball, a joystick, a touch-screen, a mouse, a switch, a dial, and the like, and any other peripheral device. In an embodiment, electrical circuitry includes one or more user input/output components that are operably coupled to at least one computing device configured to control (electrical, electromechanical, software-implemented, firmware-implemented, or other control, or combinations thereof) at least one parameter associated with, for example, determining one or more tissue thermal properties responsive to detected shifts in turn-ON voltage.

In an embodiment, electrical circuitry includes acoustic transducers, electroacoustic transducers, electrochemical transducers, electromagnetic transducers, electromechanical transducers, electrostatic transducers, photoelectric transducers, radio-acoustic transducers, thermoelectric transducers, ultrasonic transducers, and the like.

In an embodiment, electrical circuitry includes electrical circuitry operably coupled with a transducer (e.g., an actuator, a motor, a piezoelectric crystal, a Micro Electro Mechanical System (MEMS), etc.) In an embodiment, electrical circuitry includes electrical circuitry having at least one discrete electrical circuit, electrical circuitry having at least one integrated circuit, or electrical circuitry having at least one application specific integrated circuit. In an embodiment, electrical circuitry includes electrical circuitry forming a general purpose computing device configured by a computer program (e.g., a general purpose computer configured by a computer program which at least partially carries out processes and/or devices described herein, or a microprocessor configured by a computer program which at least partially carries out processes and/or devices described herein), electrical circuitry forming a memory device (e.g., forms of memory (e.g., random access, flash, read only, etc.)), electrical circuitry forming a communications device (e.g., a modem, communications switch, optical-electrical equipment, etc.), and/or any non-electrical analog thereto, such as optical or other analogs.

While illustrative embodiments have been illustrated and described, it will be appreciated that various changes can be made therein without departing from the spirit and scope of the invention.

1. A system, comprising:
a sensor system including sensors configured to obtain sensor data indicative of a user's neurophysiological response to verbal stimuli in the form of an identified emotion presented to the user and visual stimuli in the form of cosmetic colors and an avatar presented to the user via a user interface; and
a computer system including:
computational circuitry configured to cause representations of a set of available cosmetic colors to be displayed to the user via the user interface;
computational circuitry configured to receive user input indicating selection of a candidate cosmetic color from among the displayed set of available cosmetic colors;
computational circuitry configured to cause the avatar to be displayed to the user, the avatar wearing a virtual cosmetic of the candidate cosmetic color;
computational circuitry configured to receive user rating data responsive to display of the avatar wearing the virtual cosmetic of the candidate cosmetic color; and
computational circuitry configured to determine whether the candidate cosmetic color is associated with the identified emotion based on the user rating data and the sensor data.

2. The system of claim 1, wherein the user interface arranges the displayed set of available cosmetic colors in cosmetic color families or categories, and wherein the computer system further includes computational circuitry configured to receive user input indicating selection of a cosmetic color family or category.

3. The system of claim 2, wherein upon selection of the cosmetic color family or category, the displayed set of available cosmetic colors are updated such that they belong to the selected cosmetic color family.

4. The system of claim 2, wherein the cosmetic color families include red, pink, purple, orange, and brown.

5. The system of claim 1, wherein the virtual cosmetic is lipstick worn on a lip area of the avatar.

6. The system of claim 1, wherein the user interface displays to the user representations of a plurality of available avatars, and wherein the computer system further includes computational circuitry configured to receive user input indicating selection of the avatar from among the available avatars.

7. The system of claim 1, wherein the available cosmetic colors are determined by clustering colors in a database with a K-means algorithm into clusters of color patches for each of a plurality of color families.

8. The system of claim 1, wherein the sensors include brain activity sensors.

9. The system of claim 1, wherein the sensors include one or more electrodermal activity (EDA) sensors.

10. The system of claim 1, wherein the sensors include an eye tracking device configured to detect gaze direction of the user.

11. The system of claim 1, wherein the sensors include one or more heart activity sensors.

12. A method performed by a computer system, the method comprising:
receiving sensor data from sensors configured to measure a user's neurophysiological response to verbal stimuli in the form of an identified emotion presented to the user and visual stimuli in the form of cosmetic colors and an avatar presented to the user via a user interface,
displaying representations of a set of available cosmetic colors to the user via the user interface;
receiving user input indicating selection of a candidate cosmetic color from among the displayed set of available cosmetic colors displayed in the user interface;
displaying to the user the avatar wearing a virtual cosmetic of the candidate cosmetic color;
receiving user rating data responsive to display of the avatar wearing the virtual cosmetic of the candidate cosmetic color; and
determining whether the candidate cosmetic color is associated with the identified emotion based on the user rating data and the sensor data.

13. The method of claim 12 further comprising:
displaying cosmetic color families including the representations of the available cosmetic colors; and
receiving user input indicating user selection of a cosmetic color family from among the displayed cosmetic color families, wherein the displayed representations of the available cosmetic colors belong to the selected cosmetic color family.

14. The method of claim 13 further comprising, responsive to user selection of the cosmetic color family, updating the displayed set of available cosmetic colors such that each of the available cosmetic colors in the displayed set belongs to the selected cosmetic color family.

15. The method of claim 13, wherein the displayed cosmetic color families include red, pink, purple, orange, and brown.

16. The method of claim 12, wherein the virtual cosmetic is lipstick worn on a lip area of the avatar.

17. The method of claim 12 further comprising:
displaying to the user representations of a plurality of available avatars; and
receiving user input indicating user selection of the avatar from among the available avatars.

18. The method of claim 12, wherein the available cosmetic colors are determined by clustering colors in a database with a K-means algorithm into clusters of color patches for each of a plurality of color families.

19. The method of claim 12, wherein the sensors include a brain activity sensor, an electrodermal activity sensor, an eye tracking device, a heart activity sensors, or a combination thereof.

20. A makeup palette generation system comprising:
circuitry configured to generate one or more virtual objects forming part of color set, a shade set, a tint set, or a tone set on a command line user interface, a display, a graphical user interface, a menu driven user interface, a touch-sensitive display, a user input device, a user interface, or a system of interactive visual components;
circuitry configured to acquire user-specific neurophysiological data responsive to the generated one or more virtual objects forming part of color set, a shade set, a tint set, or a tone set;
circuitry configured to generate a makeup palette responsive to at least one input associated with a user-selected target emotion and the acquired user-specific neurophysiological response data; and
circuitry configured to generate a makeup simulation on an avatar based on the makeup palette generated responsive to the at least one input associated with the user-selected target emotion and the acquired user-specific neurophysiological response data.
